(19) United States
(12) Patent Application Publication
(10) Pub. No.: US 2022/0167857 A1
(43) Pub. Date: Jun. 2, 2022

(54) INTELLIGENT PORTABLE MEDICAL INSTRUMENT

(71) Applicant: He Lin, Frisco, TX (US)

(72) Inventor: He Lin, Frisco, TX (US)

(21) Appl. No.: 17/106,153

(22) Filed: Nov. 29, 2020

(51) Int. Cl. A61B 5/0205; H04Q 9/00; A61B 5/369; A61B 5/318; A61B 5/145; A61B 5/05; A61B 5/02; A61B 5/07; A61B 5/346; A61B 5/00; A61B 5/11; A61B 5/339; G16H 40/67; G16H 10/65; G16H 50/30; G16H 70/60; G16H 50/70; G16H 50/20

(52) U.S. Cl. CPC A61B 5/02055; A61B 5/022; A61B 5/369; A61B 5/318; A61B 5/14532; A61B 5/05; A61B 5/02007; A61B 5/14503; A61B 5/07; A61B 5/346; A61B 5/7275; A61B 5/0002; A61B 5/1107; A61B 5/339; G16H 40/67; G16H 10/65; G16H 50/30; G16H 70/60; G16H 50/70; G16H 50/20; H04Q 2209/84; H04Q 9/00

(57) ABSTRACT

An intelligent portable medical instrument has an information processing unit and a data storage unit which are connected to a measurement and human body data collection unit. The measurement and human body data collection unit measures electrical, chemical, and acoustic data and sends the data to the information processing unit. The information processing unit compares the measured human physiological index data with the standard ranges of values and makes a preliminary health diagnosis opinion. The preliminary health diagnosis opinion and the measured data are transmitted to an in vitro unit which preferably uploads the information to a cloud server. The in vivo portion of the intelligent portable medical instrument is provided by a single integrated circuit.

### CROSS-REFERENCE TO RELATED APPLICATION

The present application is related to Chinese Application No. 201910154788.4.

### TECHNICAL FIELD

The present invention relates to the field of medical device technology, and in particular to an intelligent portable medical instrument.

### BACKGROUND

For people who are at home, going out or traveling, the basic health monitoring and medical security devices are available only in separate thermometers, sphygmomanometers, electrocardiographs, etc. There are no portable integrated intelligent and convenient health monitoring and medical instruments available on the market which could cover all basic health situation monitoring requirements including body temperature, blood pressure, glucose, and the brain.

### SUMMARY

An intelligent portable medical instrument has a central information processing unit, a data storage unit, and a measurement and human body data collection unit which includes a set of intelligent electrical, chemical, and acoustic sensors. The central information processing unit is connected with the measurement and human body data collection unit and the data storage unit. The measurement and human body data collection unit is configured to collect human physiological indicator data and send it to the central information processing unit which stores the data in the data storage unit. The data storage unit is configured to store both the human physiological indicator data and an index of standard range values for the collected human physiological indicator data. The central information processing unit compares the collected human physiological indicator data with the standard range values and makes a preliminary health diagnosis opinion. The collected physiological indicator data and the preliminary health diagnosis opinion are passed to a remote communication module and to a cloud server. The intelligent portable medical instrument has the characteristics of intelligent monitoring and high efficiency, and is formed of integrated circuits to provide advantages of small volume and convenient carrying size.

### DESCRIPTION OF THE DRAWINGS

For a more complete understanding of the present disclosure and the advantages thereof, reference is now made to the following description taken in conjunction with the accompanying drawings in which FIGS. 1 through 17 show various aspects of the present disclosure as set forth below:

FIG. 1 is a block diagram of an intelligent portable medical instrument made according to the present disclosure which is connected to a cloud server;

FIG. 2 is a block diagram of a measuring, monitoring, and human body data collection unit of the intelligent portable medical instrument;

FIG. 3. is a block diagram of a body temperature sensor unit;

FIG. 4 is a block diagram of a blood pressure sensor unit;

FIG. 5 is a block diagram of a blood pressure sensor;

FIG. 6 is a block diagram of a biological brain electrical sensor unit;

FIG. 7 is a block diagram of the biological brain electrical sensor;

FIG. 8 is a block diagram of a bio-cardiac sensor unit;

FIG. 9 is a block diagram of the bio-cardiac sensor;

FIG. 10 is a block diagram of a first biochemical sensor unit;

FIG. 11 is a block diagram of a second biochemical sensor;

FIG. 12 is a block diagram of an acoustic wave sensor;

FIG. 13 is a top view and FIG. 14 is a sectional view taken along section line 14-14 of a block diagram of an in vivo sensor biologic sensor unit enclosed in metal packaging;

FIG. 15 is a top view and FIG. 16 is a sectional view taken along section line 16-16 of a block diagram representing an in vivo sensor biologic sensor unit enclosed in dielectric packaging 274; and

FIG. 17 is a flow chart depicting operation of the intelligent portable medical instrument of the present disclosure.

### DETAILED DESCRIPTION

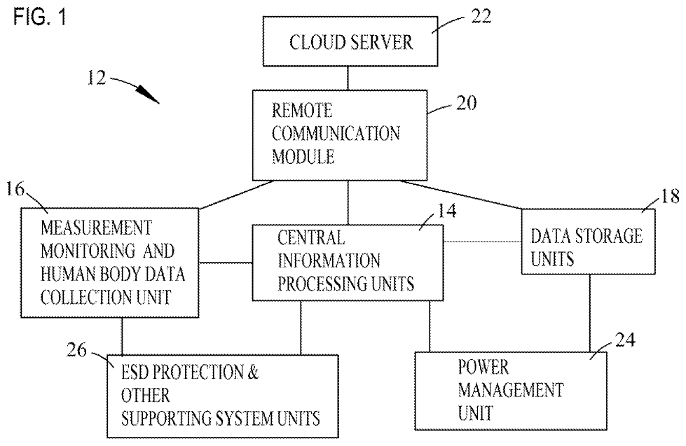

FIG. 1 is a block diagram of an intelligent portable medical instrument 12 for solving the defects of the prior art health monitoring and medical protection instruments which did not have integrated intelligence and a convenient carrying size. The invention provides an intelligent portable medical instrument 12, comprising: one or more central information processing units 14, a measurement and human body data collection unit 16 which is using multiple innovative integrated electrical, optical, acoustic, pyroelectric as well as millimeter wave sensors, and a data storage unit 18. The central information processing unit 14 and the measurement and human body data collection unit 16 and the data storage unit 18 are respectively connected. The measurement and human body data collection unit 16 is configured to collect human physiological index data and send the collected data to a central information processing unit 14 and the data storage unit 18. The data storage unit 18 is configured to store human physiological indicator data and to store preset, standard range values for human physiological indicators. The portable medical device 12 further comprises one or more electrostatic protection units 26 and a power management unit 24. The electrostatic protection unit 26 and the power management unit 24 are connected to the central information processing unit 14. The electrostatic protection unit 26 will provide static protection for key components to ensure that the portable medical instrument 12 works without any external environmental interference from lightning or human body static electricity, and the like. The power management unit 24 will power the portable medical instrument 12, providing the power required for each part, including special power requirements such as wireless power source, microwave transmitting and receiving power, and sensor power. A remote communication module 20 is provided for sending and receiving data between the portable medical device 12 and a cloud computing server 22. The remote communication module 20 may be provided with pre-amplifier, AD/DA converter circuits, and information processing and data transmission circuitry. The remote communication module may receive and send data either wirelessly (through RF transmission) or use a conventional wired connection to the cloud server.

The intelligent portable medical instrument 12 collects various human physiological index data in real time through the measurement and human body data collecting unit 16 and sends it to the central information processing unit 14 and the data storage unit 18. The data storage unit 18 is preferably used for storing both the measured human physiological data and the standard range values of the human physiological index data. The central information processing unit 14 compares the measured human physiological index data with the standard range values of the human physiological index data and makes a preliminary health diagnosis opinion based on the analysis result. The health diagnosis opinion is sent to the cloud server 22 through the remote communication module 20. The portable medical instrument 12 integrates multiple human physiological index detection functions into one instrument by using an advanced large-scale integrated circuit and uses the central information processing unit 14 to analyze and obtain preliminary health diagnosis opinions. The portable medical instrument 12 provides real-time monitoring of human health, monitoring various human physiological index data with intelligence and high efficiency. An integrated circuit is preferably used for all major sections of the intelligent portable medical instrument 12 to provide a small size which is convenient to carry.

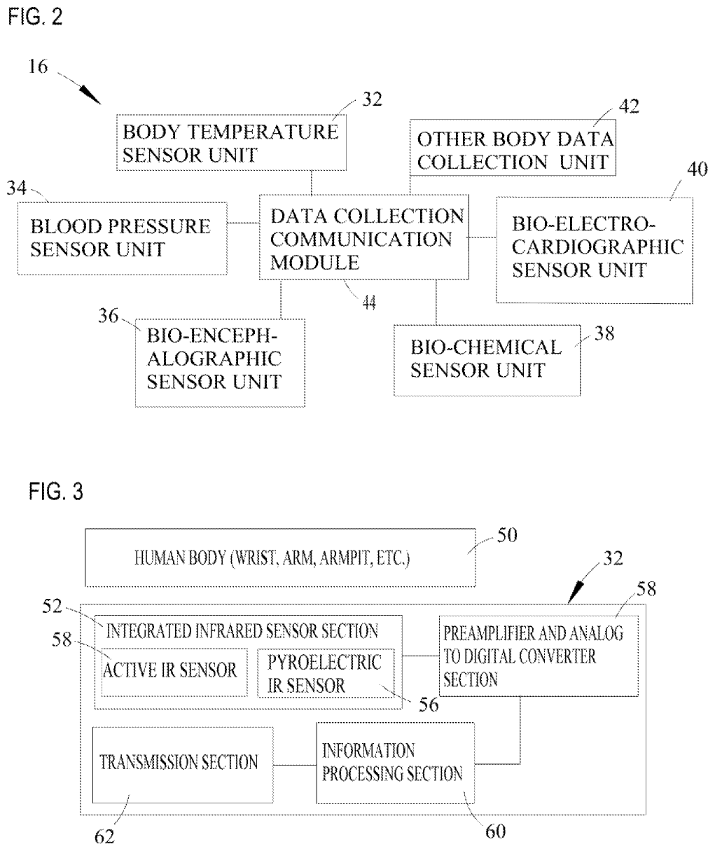

FIG. 2 is a block diagram of the human body data collection unit 16 of the intelligent portable medical instrument 12. The measuring and human body data collecting unit 16 includes, but is not limited to: one or more of a body temperature sensor unit 32, a blood pressure sensor unit 34, a biological brain electrical sensor unit 36 (bio-encephalographic sensor), a biochemical sensor unit 38, and a bio-cardiac sensor unit 40, and other body data measurement and collections units 42. The body temperature sensor 32 is preferably provided by an in vivo infrared temperature sensor which in implanted into the body. The blood pressure sensor unit 34 is preferably an in vivo pressure sensor which is implanted into the body. The bio-encephalographic sensor unit 36 is preferably provided by an in vivo multi-terminal brain-electrical sensor unit, and an in vivo multi-terminal pressure sensor. The bio-chemical sensor unit 38 is preferably provided by an in vivo multi-end biochemical sensor, such as for detecting blood sugar, blood sodium, blood potassium sensor, and the like. The bio-cardiac sensor unit 40 is preferably an in vivo multi-end bio-electrocardiogram, a pressure sensor, and an in vivo multi-terminal electrocardiographic sensor. The other body data collection unit 42 preferably includes sensors for detecting blood sugar, blood sodium, blood potassium, electrocardiogram, and other body data for real-time monitoring of human health. The various sensor units 32-42 are connected to a data communication module 44, which interfaces between the sensor units 32-42 and the central information processing unit 14 (shown in FIG. 1). Each sensor unit 32-42 preferably includes supporting circuitry such as a pre-amplifier, an AD/DA converter, information processing and data transmission, and a power management unit. Data may be transmitted between the data communication module 44 and the various sensor units 32-42 either wirelessly (RF transmission) or by means of a conventional hard wire connection. The data communication module 44 may also be connected to the central information processing unit 14 either wirelessly or by a hard-wired connection.

FIG. 3 is a block diagram of a body temperature sensor unit 32. The body temperature sensor 32 comprises an integrated infrared sensor section 52 which has both an active optical IR sensor 54 and a pyroelectrical IR sensor 56. The active IR sensor 54 is configured for detecting IR wavelength ranges from 8 to 14 microns which is the peak wavelength from human body radiation. The pyroelectrical IR sensor 56 will be used to cover a wider infrared radiation range, such as from 0.8 to 20 microns. The integrated IR sensor section 52, including both the active IR sensor 54 and the pyroelectrical IR sensor 56, will be configured from superlattice based III-V semiconductor compound devices, such as GaN, GaAlN, InAs, and the like. Use of both the active IR sensor 54 and the pyroelectric IR sensor 56 increases the body temperature accuracy and body temperature distribution. The sensor unit 32 further includes a preamplifier and an Analog to Digital (AD) conversion section 58, an information processing section 60, and a signal transmission section 62. All the above sections are preferably integrated into one semiconductor integrated circuit chip which will make the device very small and convenient for portable applications.

The body temperature sensor unit 32 may be provided in different types of packages, such as being wearable on the wrist, wearable on the arm, carried by hand, or mounted to an armpit or ear, and such. The body temperature signals obtained from both active optical IR sensor 54 and the pyroelectric sensor 56 will be input to a low noise preamplifier and then the output analog signal will be converted to the digital signal in the section 58. The digital signal from the section 58 will be passed to the information processing section 60 and then to the transmission section 62. The transmission section 62 will combine the digital signal with a carrier RF transmission signal. The RF transmission signal will be received by the wireless RF receiver in the external information receiving unit 20 (shown in FIG. 1) in which the digital signal will be separated from the carrier RF transmission signal and then passed to the central signal processing unit 14 (shown in FIG. 1).

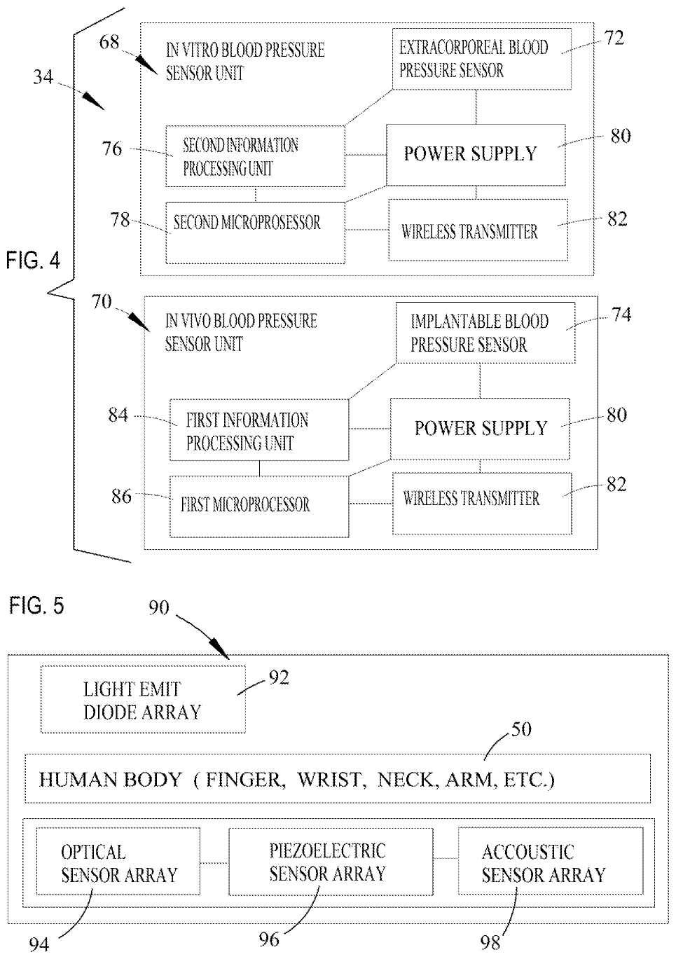

FIG. 4 is a block diagram of a blood pressure sensor unit 34 which includes an internally disposed, in vivo blood pressure sensor unit 70 and an externally disposed, in vitro blood pressure sensor unit 68. Preferably both the in vivo blood pressure sensor unit 70 and the in vitro blood pressure sensor unit 68 will automatically measure blood pressure using Pulse Transit Time (PTT) methodology. The in vitro blood pressure sensor unit 68 includes an in vitro blood pressure sensor 72, a second information processing unit 76, a second microprocessor 78, a power supply unit 80 and a wireless transmitter 82. The in vivo blood pressure sensor unit 70 includes an in vivo blood pressure sensor 74, a first information processing unit 84, a first microprocessor 86, a power supply unit 80 and a wireless transmitter 82. The unit is connected to the in vivo first pressure sensor to receive and process blood pressure information of the human body. The in vitro blood pressure sensor 72 and the in vivo first pressure sensor 74 are both configured to collect human blood pressure information, preferably using the Pulse Transit Time (PTT) methodology. An innovative approach is used for realizing the PTT method for the automatic blood pressure measurement.

The in vitro blood pressure sensor 72 is located outside the human body and has an input end which preferably directly contacts a human body to collect blood pressure information, as discussed below in reference to FIG. 4. The in vitro blood pressure sensor 72 has an output end which transmits the blood pressure information to the second information processing unit 76, which then transmits the processed human blood pressure information to the second microprocessor 78. The second microprocessor 78 then passes the blood pressure information to the wireless transmitter 82, and then the information is transmitted to a wireless receiver and to the central information processing unit 14 of FIG. 1. The power supply unit 80 is connected to the in vitro pressure sensor 72, the second information processing unit 78, the second microprocessor 78 and the wireless transmitter 82. The power supply unit 80 is preferably provided by a plurality of micro batteries. The in vivo blood pressure sensor 74 is preferably a miniature pressure sensor. The first information processing unit 76 is preferably an amplifier.

The in vivo blood pressure sensor 74 is located inside the human body and has an input end which preferably directly contacts a human blood vessel to collect blood pressure information of the human body. The in vivo blood pressure sensor 74 has an output end which transmits the blood pressure information to the first information processing unit 84, which then transmits the processed human blood pressure information to the first microprocessor 86. The first microprocessor 86 passes the blood pressure information to the wireless transmitter 82 and then the information is transmitted to a wireless receiver outside the human body. The power supply unit 80 is connected to the in vivo pressure sensor 74, the first information processing unit 84, the first microprocessor 86 and the wireless transmitter 82. The power supply unit 80 is preferably provided by a plurality of micro batteries. The in vivo blood pressure sensor 74 is preferably a miniature pressure sensor. The first information processing unit 84 is preferably an amplifier.

FIG. 5 is a block diagram of a blood pressure sensor, which shows the working principle of the blood pressure measurement using Pulse Transit Time (PTT) methodology. A wearable blood pressure measurement unit 90 consists of a light emitting diode array 92, an optical sensor array 94, a piezoelectric device 96 and an acoustic sensor array 98; and preferably each are integrated together into one single semiconductor chip which will greatly improve the reliability and accuracy of measurements. During the blood pressure data collection, one or two fingers of a human body 50 will be put between the light emitting diode array 92 and the optical sensor array 94. The light emitting diode array 92 will emit light in the wavelength range of visible and near infrared. Like conventional Photoplethysmography (PPG) methodology, this method could detect blood volume changes in the microvascular of human tissue. To improve the blood pressure measurement accuracy and reliability, a piezoelectric sensor array 96 can both detect acoustic waves and can generate acoustic waves. The piezoelectric sensor array 96 will be placed at the surface of the human finger, wrist, arm or other places which could detect the minor change of the surface pressure of vascular and microvascular systems, the blood pressure related data, such as blood volume data collected by the PPG method. Additionally, an acoustic sensor array 98, separate from the piezoelectric sensor array 96, will be placed against the body. The pulse pressure data from the piezoelectric sensor array 96 and the acoustic sensor array 98 will be collected from at least two wearable devices, such as one worn on a user's wrist and one worn on a user's finger, respectively. The Pulse Transit Time (PTT) can be derived from those data, especially the time difference information between devices at different locations, for instance the distance between the wearable device worn on the wrist and the wearable device worn on the finger.

The optical sensor array will be provided by a superlattice photo detector array which could have multiple designs. A typical design consists of a substrate, a transition layer over the substrate, an intrinsic semiconductor superlattice layer, a N-type semiconductor superlattice layer, the second intrinsic semiconductor superlattice layer, and a P-type semiconductor superlattice layer. The optical sensor preferably works in an active mode, triggered by an LED or laser diode and generating bio-signal responses by detecting the reflection and diffraction from an LED or Laser diode. A specially designed package with an optical window will be used for the device with the optical sensor array. The material selection of the optical window of the package will depend on the optical wavelength range of the LED or Laser diode. The piezoelectric sensor array 96 and the acoustic array 98 are preferably provided by superlattice sensor arrays formed on a structure that is using GaN or other semiconductor material which has a wurtzite structure. This material and structure provide very strong piezoelectric, pyroelectric, and acoustic effects so that both piezoelectric sensor and acoustic sensor could be made using a GaN type superlattice structure. A typical GaN superlattice piezoelectric sensor could be made by following major steps: depositing a transition layer on a semiconductor substrate, growing superlattice thin layers over the transition layer, then depositing GaN layer on the top as piezoelectric sensing layer. Preferably a HEMT (High Electron Mobility Transistor) type of transistor will be made to detect any pressure related piezoelectrical signal using the transistor structure. A novel GaN superlattice acoustic sensor could be made by the following steps: depositing a transition layer on a semiconductor substrate, growing GaN acoustic layer over the transition layer, and then depositing GaN layers on the top as acoustic signal sensing layer. Preferably an HEMT (High Electron Mobility Transistor) type of transistor will be made to detect any acoustic related signal using the transistor structure. A SAW (Surface Acoustic Wave) type of structure could also be produced using GaN type material for the medical sensing application.

The major differences between the piezoelectric sensor array 96 and the acoustic sensor array 98 are the following: (a) The piezoelectric sensor array 96 utilizes the piezoelectrical material as the gate for the HEMT transistor, the human blood pressure changing generated piezoelectric signal would result in the modulation of the two dimensional electrons and holes of the HEMT transistor, but the acoustic sensor array 98 utilizes structures, such as cavity type structures to catch the acoustic signals associated to the blood pressure change; (b) the piezoelectric sensor array 96 is provided by depositing thin layers of piezoelectrical material (typically around 100 to 300 nanometers thick) while the acoustic layer deposited for the acoustic sensor array 98 is thicker, with thicknesses up to and over one micron; (c) an acoustic cavity might be required in some acoustic sensors, such as for a SAW (Surface Acoustic Wave) devices. A unique packaging approach is used for the piezoelectrical and acoustic sensor arrays: The packaging will leave a window for the sensing area of the piezoelectrical and acoustic sensor array, but other areas on the packaging will be sealed from the exterior of the packaging.

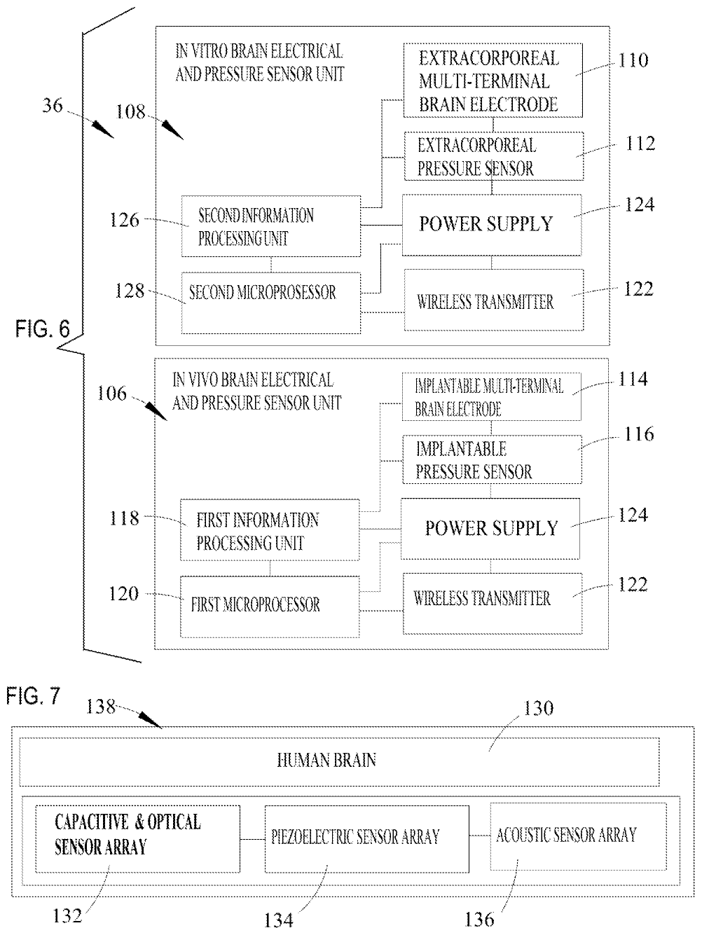

FIG. 6 is a block diagram of a biological brain electrical sensor unit 36 which is composed of an in vivo biological brain electrical sensor unit 106 and an in vitro biological brain electrical sensor unit 108. The in vivo bio-encephalographic sensor unit 106 comprises an in vivo multi-terminal EEG electrode 114 and an in vivo second pressure sensor 116. The in vivo multi-terminal brain electrical electrode 114 is configured for collecting human brain electrical information, and the in vivo second pressure sensor 116 is configured to measure the contact pressure at which the in vivo multi-terminal brain electrical electrode 114 is pressed against the human body. Preferably, the electrode contact pressure is that which provides accurate electrical readings while comfortable to the human body. The in vivo multi-terminal EEG electrode 114 and the implantable second pressure sensor 116 are connected to a first information processing unit 118 and then to a first microprocessor 120. The first microprocessor 120 is connected to a wireless transmitter 122 for transmitting the measured date external of the body and to the central processing unit 14. A power supply 124 provides power to the implanted sensor unit 106.

The in vitro bio-encephalographic sensor unit 108 comprises an in vitro multi-terminal EEG electrode 110 and an in vitro pressure sensor 112. The in vitro multi-end EEG electrode 110 is used for collecting human brain electrical information, and the in vitro pressure sensor 112 is used for collecting measurements of the contact pressure of the in vitro multi-end EEG electrodes 110 against the human body. The contact pressure is preferably a contact pressure that is comfortable to the human while allowing for accurate electrical readings. The in vitro multi-terminal EEG electrode 110 and the in vitro second pressure sensor 112 are connected to a second information processing unit 126 and then to a second microprocessor 128. The second microprocessor 128 is connected to a wireless transmitter 122 for transmitting the measured data to the central processing unit 14. A power supply 124 provides power to the extracorporeal sensor unit 108.

FIG. 7 is a block diagram of a biological brain electrical sensor 138 which includes a capacitive and optical sensor array 132, a piezoelectric sensor array 134, and an acoustic sensor array 136 which are preferably integrated into a single semiconductor integrated circuit chip. The electric magnetic signal generated from a human brain 130 may range from a few hertz to a few hundred hertz, which means the wavelength will mainly in the acoustic range. The capacitive and optical sensor array 132 will cover the biological electric data of a human brain 130 from micro to millimeter range which is critical to detect any impact on the human brain from external radiation, while the acoustic sensor will cover the typical human brain signal from a few hertz to a few hundred hertz, such as around 8 hertz to 500 hertz range. The piezoelectric sensor array 134 will collect the surface pressure of vascular and microvascular systems for the brain. The acoustic sensor array 136 will also collect surface pressure of the vascular and microvascular systems for the brain 130.

The Capacitive Sensor array could be made in different ways, using the bioelectrical charging effect of the human body through a specially designed semiconductor structure by either sensing a human body generated bio-electrical field or by sensing changes in an external electrical field applied to the body. Under either method, a detected capacitive signal would be proportional to the bioelectrical charging. A typical bioelectrical capacitive sensor could be made using Superlattice bioelectronic impedance sensors which include: a superlattice intrinsic layer, a superlattice P-type layer, a second superlattice intrinsic layer, a superlattice N-type layer, a P+ conductive layer, a gate insulation layer, an ohmic contact layer, a dielectric protection layer, a channel insulation layer and a biological medium layer. The superlattice intrinsic layer, the superlattice P-type layer, a second superlattice intrinsic layer, the superlattice N-type layer, the P+ conductive layer, the gate insulation layer, the ohmic contact layer, the dielectric protection layer and the channel insulation layer are symmetrically distributed on both sides of the bio-media layer. The ohmic contact layer includes a source, a drain, a first gate, and a second gate. The source and drain are symmetrically distributed on both sides of the bio-media layer. The first grid and the second grid are symmetrically distributed on both sides of the biological medium layer. The optical sensor array will be provided by a superlattice photo detector array which could have multiple designs. A typical design would consist of a substrate, a transition layer over the substrate, an intrinsic semiconductor superlattice layer, a P-type semiconductor superlattice layer, the second intrinsic semiconductor superlattice layer, and an N-type semiconductor superlattice layer. Both capacitive and optical sensor could work either in passive mode or in active mode as active sensors. For active modes, the capacitive sensor could be triggered by an LED or laser diode (Photo Capacitance Mode), and the optical sensor could generate bio-signal responses by detecting the reflection and diffraction from an LED or a Laser diode.

For in vivo applications, the special package is designed by sealing the intelligent medical chip by inert materials such as SiO2, SiN, SiC , etc. except for the bioelectrical capacitive sensor. The unique designed packaging methods are used for the optical sensor array. For the optical sensor array which is working in the wavelength from UV to mid-infrared range, such as from 0.3 to 3.5 μm, the quartz material could be a good candidate. As noted above in reference to FIG. 5, the piezoelectric sensor array 134 and the acoustic sensor array 136 are preferably provided by superlattice sensor arrays formed on a single GaN semiconductor material which has a wurtzite structure. Other semiconductor materials may be used such as AlN, AlP, or the like. This material and structure have very strong piezoelectric, pyroelectric, and acoustic effects so that both piezoelectric sensor and acoustic sensor could be made using a GaN type superlattice structure. A typical GaN superlattice piezoelectric sensor could be made by following major steps: depositing a transition layer on a semiconductor substrate, growing superlattice thin layers over the transition layer, then depositing GaN layer on the top as piezoelectric sensing layer. Preferably a HEMT (High Electron Mobility Transistor) type of transistor will be made to detect any pressure related piezoelectrical signal using the transistor structure. A novel GaN superlattice acoustic sensor could be made by following major steps: depositing a transition layer on a semiconductor substrate, growing GaN acoustic layer over the transition layer, and then depositing GaN layers on the top as acoustic signal sensing layer. Preferably an HEMT (High Electron Mobility Transistor) type of transistor will be made to detect any acoustic related signal using the transistor structure. A SAW (Surface Acoustic Wave) type of structure could also be produced using GaN type material for the medical sensing application.

Proper package designs are used for piezoelectric sensor array and acoustic wave sensor array, etc. For in vivo application, a dielectric package using materials with very stable chemical and temperature characteristics, such as SiO2 (Quartz type), SiN, SiC, or metal, such as Titanium, could be used. An open window may be provided to expose the sensing areas of piezoelectric sensor array and acoustic wave sensor array.

FIG. 8 is a block diagram of a bio-cardiac sensor unit 36 which is composed of an in vivo bio-electrocardiographic sensor unit 144 and an in vitro bio-electrocardiographic sensor unit 146. The in vitro bio-electrocardiographic sensor unit 146 includes an in vitro multi-terminal ECG electrode 148 and an in vitro pressure sensor 150 which are connected together for collecting both ECG information and the contact pressure at which the multi-terminal electrocardiographic electrode 148 is pressing against the human body. Preferably contact pressure is at a level for obtaining accurate readings while also being comfortable to the human body.

The in vitro multi-terminal ECG electrode 148 and the in vivo pressure sensor 150 are connected to a second information processing unit 164, which is connected to a second microprocessor 166. The information processing unit 164 receives and processes the human body ECG information and the pressure information, and preferably is provided by an amplifier and may include signal conditioning circuitry and one or more analog to digital converters. A data signal is passed from the second information processing unit 164 to the second microprocessor 166. The microprocessor 166 is also connected to the in vivo multi-terminal ECG electrode 166 for controlling operation of the in vitro multi-terminal ECG electrode 148 to collect the extracorporeal ECG signal. A power supply 162 provides electrical power to the in vitro electrocardiographic and pressure sensor unit 146, and a wireless transmitter is connected to the microprocessor 166 for transmitting collected data to the central processing unit 14 (shown in FIG. 1).

The in vivo bio-electrocardiographic sensor unit 144 includes an in vivo multi-terminal ECG electrode 152 and an in vivo pressure sensor 154. The in vivo multi-terminal electrocardiographic electrode 152 is configured to collect human body electrocardiographic information. The in vivo pressure sensor 154 is configured to collect the contact pressure at which the in vivo multi-terminal electrocardiographic electrode 152 is pressing against the human body contact portion. The in vivo multi-terminal ECG electrode 152 and the in vivo pressure sensor 154 are connected to a first information processing unit 156, which is connected to a first microprocessor 158. The information processing unit 156 receives and processes the human body ECG information and the pressure information, and preferably is provided by an amplifier and may include signal conditioning circuitry and one or more analog to digital converters. A data signal is passed from the first information processing unit 156 to the first microprocessor 158. The microprocessor 158 is also connected to the in vivo multi-terminal ECG electrode 152 for controlling operation of the in vivo multi-terminal ECG electrode 152 to collect the ECG signal. A power supply 162 provides electrical power to the in vivo electrocardiographic and pressure sensor unit 144, and a wireless transmitter is connected to the microprocessor for transmitting collected data to the central processing unit 14 (shown in FIG. 1).

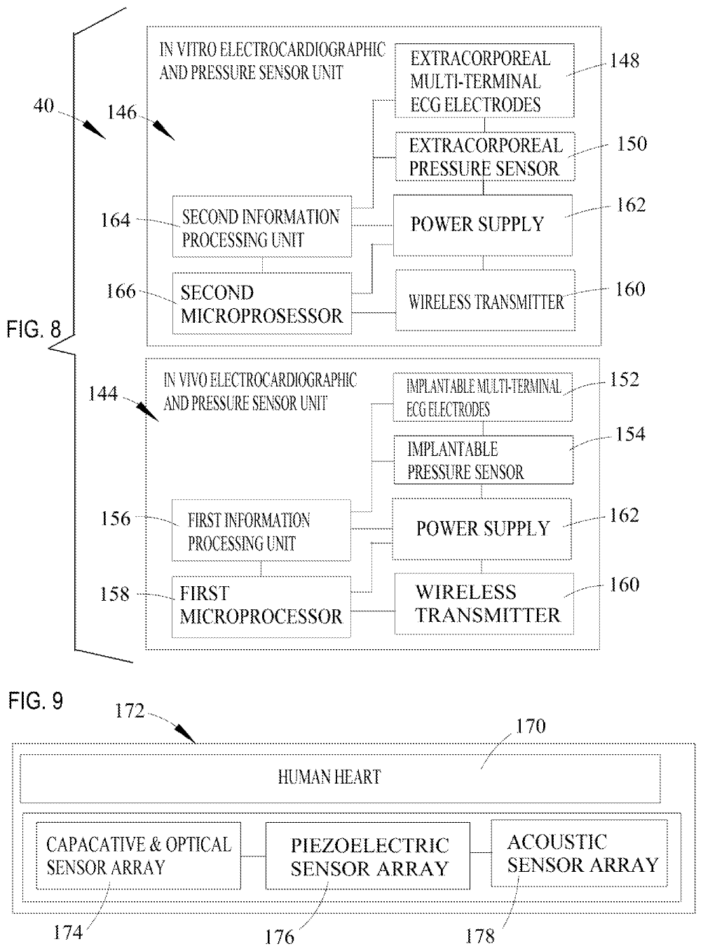

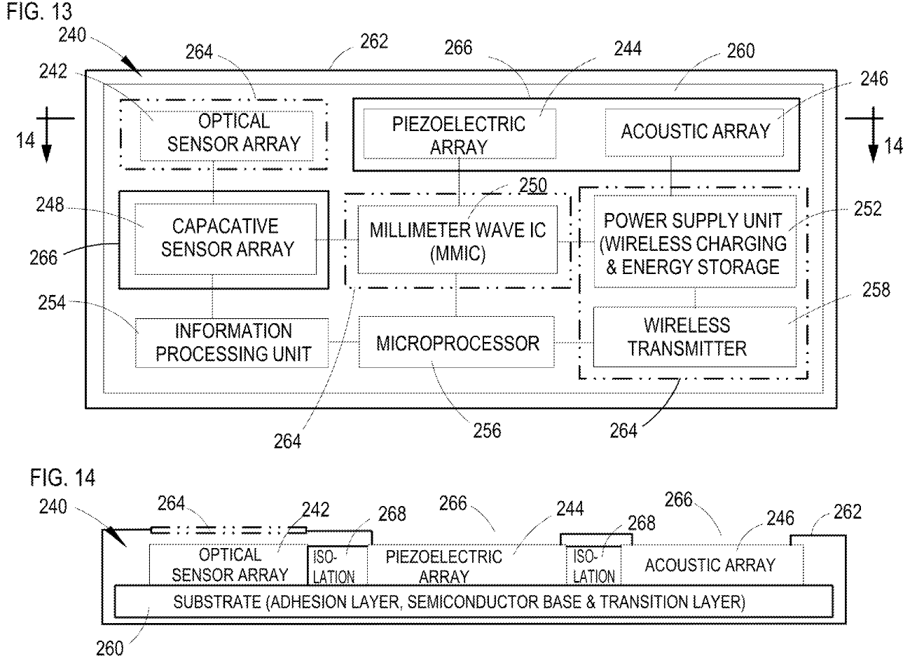

FIG. 9 is a block diagram of a bio-cardiac sensor 172 which includes a capacitive and an optical sensor array 174, a piezoelectric sensor array 176 as well as an acoustic sensor array 178 which have been integrated together into one semiconductor integrated circuit chip. The capacitive and piezoelectric sensor array 174 are used to catch the conduction signals from the heart, such as transmit signals of the sinoatrial node (SA) and atrioventricular node (AV node), etc. The piezoelectric sensor array 176 detects and measures pressure variations emanating from the heart 170. The acoustic sensor array 178 is utilized to detect critical acoustic signals from the heart 170, such as the heartbeat rate (BPM—Beats Per Minute), characteristics of coronary artery, cardiac muscle contraction, etc. To increase the detection sensitivity, an acoustic radar system such as that shown in FIG. 12 may be included in the detection complex. As noted above in reference to FIGS. 5 and 7, the capacitive and optical sensor array 174, the piezoelectric sensor array 176, and the acoustic sensor array 178 are preferably provided by superlattice sensor arrays formed on a semiconductor substrate using GaN type of semiconductor material which has a wurtzite structure. Other semiconductor materials such as AlN or AlP may also be used.

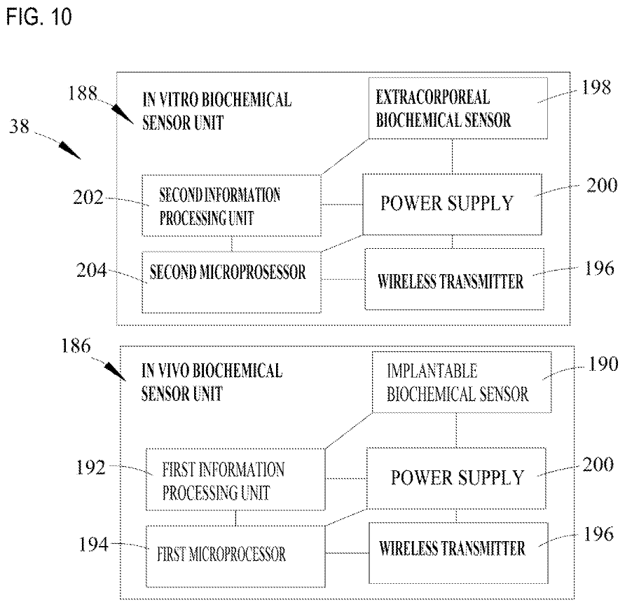

FIG. 10 is a block diagram of a first biochemical sensor unit 38 which includes an in vivo biochemical sensor unit 186 and an in vitro biochemical sensor unit 188. The in vitro biochemical sensor unit 188 includes an in vitro multi-end biochemical sensor 198 which collects biochemical information of the human body, such as blood sugar, blood sodium, blood potassium, and the like. The in vitro multi-end biochemical sensor 198 is coupled to a second information processing unit 202, which is connected to a second microprocessor 204. The second microprocessor 204 is connected to the transmitter 196 for transmitting the collected data to the central processor 14 of FIG. 1. A power supply 200 provides electric power to the in vitro biochemical sensor unit 188. The in vivo biochemical sensor unit 186 includes an in vivo multi-end biochemical sensor 190 which also collects biochemical information of the human body, such as blood sugar, blood sodium, blood potassium, and the like. The in vivo multi-end biochemical sensor 190 is coupled to a first information processing unit 192, which is connected to a first microprocessor 194. The first microprocessor 194 is connected to the transmitter 196 for transmitting the collected data to the central processor 14 of FIG. 1. A power supply 200 provides electric power to the in vivo biochemical sensor unit 186.

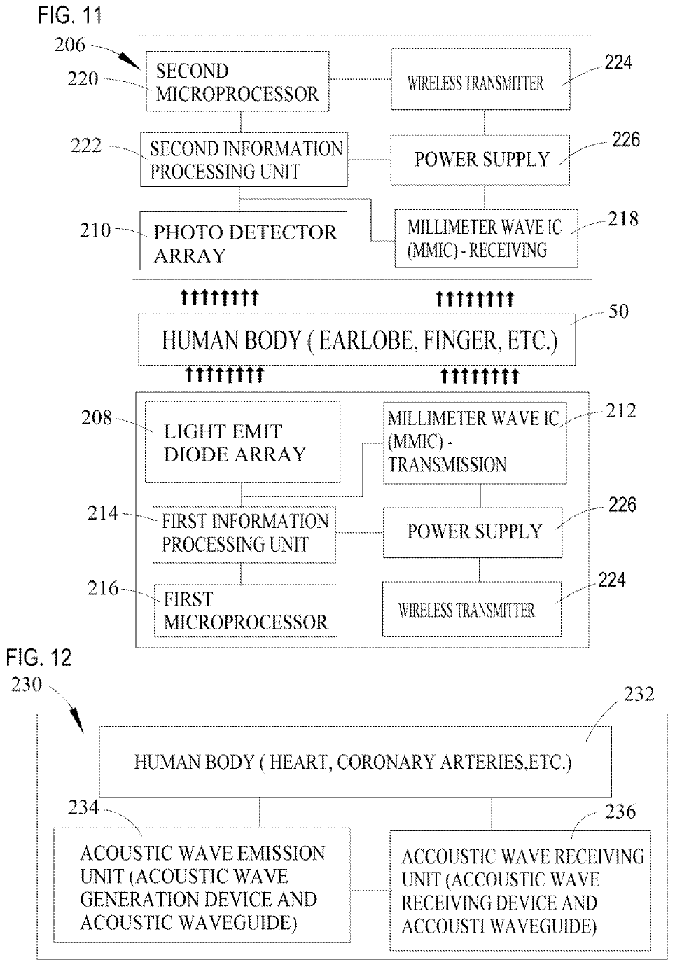

FIG. 11 is a block diagram of a second biochemical sensor unit 206 providing a blood glucose sensor which is a typical application for the biochemical sensor. The biochemical sensor unit 206 includes an intelligent signal source chip containing a light emitting diode (LED) array 208 which is covering wide optical spectrum range from UV to infrared and a millimeter wave integrated circuit (MMIC) 212 for transmission of millimeter wave radio frequencies. A first information processing unit 214 provides signal conditioning and processing, together with the central microcontroller 216, for the light emitting diode array 208 and the millimeter wave IC transmission unit 212. A power supply unit 226 provides power management. A wireless transmitter 224 is provided for sending and receiving data signals from the sensor unit 206. The sensor unit 206 also includes a millimeter wave receiving integrated circuit (MMIC) 218. A photo detector array 210 is provided with wide spectrum optical sensor array which is covering the detection range from UV to infrared. The portion of the human body 50, such as earlobe, finger, hand, etc., would be placed between an intelligent signal source, such as the light emitting diode array 208 and the millimeter wave IC transmission unit 216, and photo detector array 210 and the millimeter wave IC receiving unit 218, respectively. The optical and millimeter wave signal that is passing through human body 50 will be received by the photo detector array 210 and the millimeter wave IC receiving unit 218. The photo detector array 210 and the millimeter wave signal receiving unit 218 both connect to a second information processing unit 222 having signal preamplifiers, AD (Analog To Digital) & DA (Digital To Analog) converters, and other signal conditioning circuits. The second information processing unit provides digital output signals to the second microprocessor 220. A power supply 226 provides both a power supply and power management. A wireless transmitter 224 is connected to the second microprocessor 220 for communicating with vitro communication units.

FIG. 12 is a block diagram of an acoustic wave sensor 230. The acoustic wave sensor 230 has an acoustic wave emission unit 234 which includes both an acoustic wave generation device and an acoustic waveguide for transmitting acoustic signals to the human body 232. The acoustic wave sensor 230 also includes an acoustic wave receiving unit 236 having an acoustic wave receiving device coupled to an acoustic waveguide. The acoustic wave emission unit 234 will emit acoustic signals which are passed through a human body 232 and received by the acoustic wave receiving unit 236. Attenuation of the acoustic signals sent there-between are indicative of various biological and chemical parameters. A typical wurtzite material like GaN, AlN, or such, formed as a film may be utilized to both generate piezoelectric signals and receive the returned acoustic signals. The acoustic devices may also be integrated into the same chip, with one portion designed as an acoustic wave emission unit and another portion designed to be an acoustic wave receiving unit. The acoustic wave emission units and the acoustic wave receiving units will each preferably consist of an acoustic wave generation device and an acoustic waveguide. When the AC electrical bias is applied to the acoustic generation device, an acoustic wave signal is generated and emitted. The acoustic wave signal would be reflected from the part of the human body which is being monitored, and the reflected acoustic wave signal would be detected by an acoustic wave receiving unit having an acoustic wave receiving device and an acoustic waveguide.

FIGS. 13 is a top view and FIG. 14 is a sectional view taken along section line 14-14 of a block diagram representing an in vivo sensor biologic sensor unit 240 disposed in an enclosure provided by metal packaging 262. The metal packaging 262 may be provided using materials such as Titanium, or the like, with the sensing area of piezoelectric sensor arrays 244, the acoustic wave sensor arrays 246, and the capacitive sensor array 248 having open windows 266 for passing measurement signals. The in vivo biologic sensor 204 preferably has components integrated with a single substrate 260. The components are shown to include: an optical sensor array 242, a piezoelectric array 244, an acoustic array 246, a capacitive sensor array 248, a millimeter wave IC (MMIC) 250, a power supply and wireless charging unit 252, an information processing unit 254, a microprocessor 256, and a wireless transmitter 258 for communication with in vitro wireless transmitters. Preferably open windows 266 are provided in the metal packaging 262 adjacent to the piezoelectric array 244, the acoustic array 246, and the capacitive sensor array 248. Coatings which are transmissive in regard to respective type arrays 244, 246, and 248 may optionally be applied to seal the open windows 266. Preferably windows 264 formed into the metal packing 262 which are made of dielectric materials will be located adjacent to the optical sensor array 242, the millimeter wave IC (MMIC) 250, the power supply unit with wireless charging 252 and the wireless transmitter 264. The windows 264 and 266 provide for transmitting and receiving sensor signals, data signals and power charging signals from the in vivo sensor 240 disposed in the metal packaging 262. In FIG. 14 isolation sections 268 are shown disposed between adjacent ones of the arrays 242, 244 and 246.

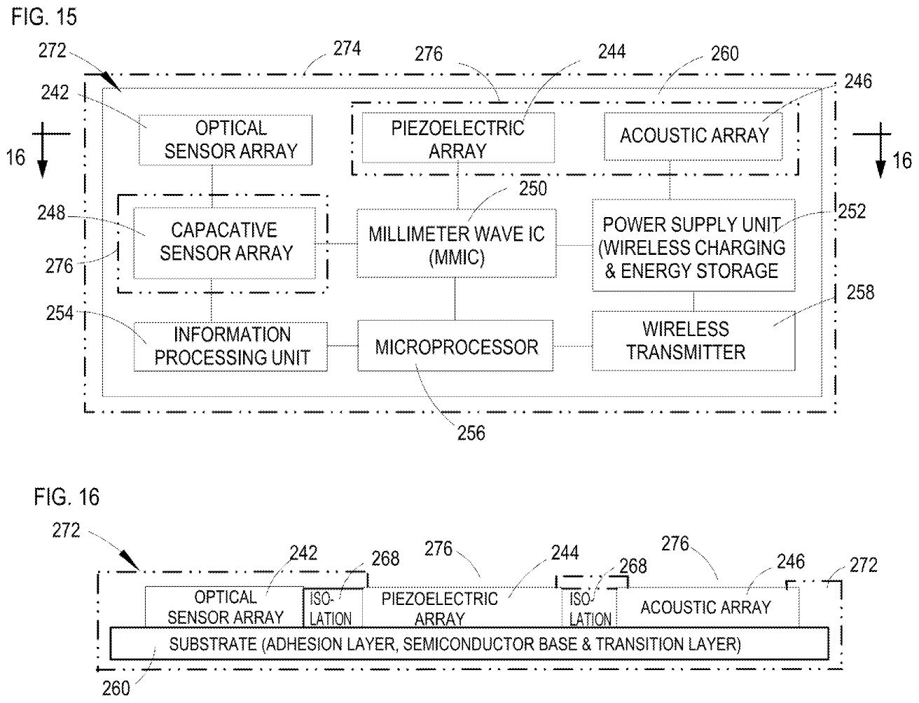

FIG. 15 is a top view and FIG. 16 is a sectional view taken along section line 16-16 of a block diagram representing an in vivo sensor biologic sensor unit 272 disposed in an enclosure provided by dielectric packaging 274. The dielectric packaging 274 may be provided with stable chemical and temperature characteristics using materials such as SiO2 (Quartz type), SiN, SiC, or such. The sensing area of piezoelectric sensor arrays 244 and acoustic wave sensor arrays 246 and the capacitive sensor array 248 having open windows 276 for passing measurement signals. The in vivo biologic sensor 272 preferably has components integrated with a single substrate 260. The components are shown are the same as those in FIGS. 13 and 14, and include: an optical sensor array 242, a piezoelectric array 244, an acoustic array 246, a capacitive sensor array 266, a millimeter wave IC (MMIC) 250, a power supply and wireless charging unit 242, an information processing unit 252, a microprocessor 256, and a wireless transmitter 264 for communication with in vitro wireless transmitters. Preferably open windows 266 are provided in the dielectric packaging 272 adjacent to the piezoelectric array 244, the acoustic array 246, and the capacitive sensor array 266. Coatings which are transmissive in regard to respective type arrays 244, 246, and 266 may optionally be applied to seal the open windows 276. In FIG. 16 isolation sections 268 are shown disposed between adjacent ones of the arrays 242, 244 and 246.

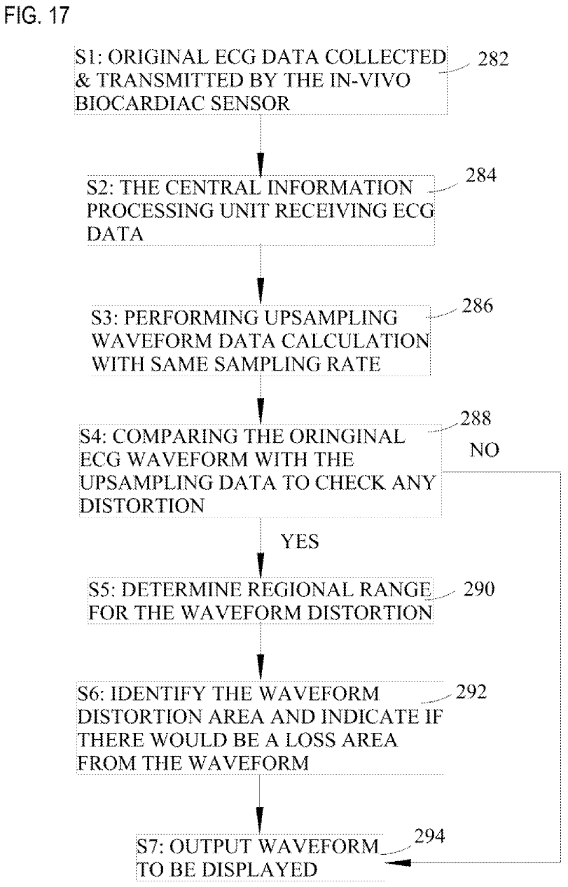

FIG. 17 is a flow chart depicting operation of the intelligent portable medical instrument of the present disclosure. Preferably, the medical device ECG signal display method comprises the following steps. In block 282, step 1, the original ECG data is collected the in vivo cardiac sensor unit 40, shown in FIGS. 2 and 8, and then is transmitted to the data collection module 44 of FIG. 2. The data collection module 44 forwards the EDG data to the central information processing unit 14, shown in FIG. 1. In block 284, step 2, the central information processing unit 14 receives the original ECG waveform data transmitted by the implanted in vivo bio cardiac sensor unit 40 and obtains waveform data to be displayed that matches the resolution of the display medium. In block 286, step 3, an up-sampling calculation is performed on the waveform data to be displayed. Up-sampled waveform data is obtained with the same sampling rate as the original electrocardiographic waveform data. In block 288, step 4, a comparison is made between the original ECG waveform data and the up-sampled waveform data, and a determination is made as to whether there is waveform distortion. If waveform distortion is detected, the process proceeds to block 290, step 5. If waveform distortion is not detected, the process proceeds to block 294, step 7. In block 290, step 5, a determination is made of the region of the waveform in which the distortion is detected. Then the process proceeds to block 292, step 6. In block 292, step 6, the waveform distortion region is identified and an indication is provided as to whether there is a loss area from the waveform. In block 294, step 7, a waveform is output to be displayed, and a waveform distortion area is identified to indicate that there is a loss area of the waveform presented at the current resolution. The quantified data for the loss area of the waveform could be obtained in comparison with the original information.

The working principle and beneficial effects of the above technical solution are as follows. The above technical solution adopts an infrared sensor to collect human body temperature information which has the advantages of convenient and fast data collection, and accurate body temperature information. The blood pressure sensor unit adopts the first information processing unit and the in vivo type pressure sensor is connected to receive and process the blood pressure information of the human body, so that the blood pressure information transmitted outside the human body is accurate. The second information processing unit of the biological brain electrical sensor unit receives and processes the human brain electrical information transmitted by the in vivo multi-end brain electrical electrode. The second contact pressure information transmitted by the in vivo second pressure sensor is such that the human brain electrical information and the second contact pressure information transmitted outside the human body are accurate. The biochemical sensor unit receives and processes the biochemical information of the human body by using the third information processing unit. The biochemical information transmitted outside the human body is accurate. The bio-energy sensor unit receives and transmits the human body electrocardiogram information and the fourth contact pressure signal outside the human body.

The above technical solutions use multiple innovative integrated electrical, optical, acoustic, pyroelectric as well as millimeter wave sensors, bio-electronics, biochemistry, artificial intelligence and other technologies to provide basic monitoring and medical support functions for human health, covering major functions of instruments, such as thermometers, sphygmomanometers, blood glucose meters and electrocardiographs. It has the characteristics of multi-function, high efficiency, intelligent design, small size, portability, and ease of transport.

In one embodiment, an in vitro blood pressure sensor unit includes a wireless receiver. The in vivo blood pressure sensor unit includes a housing which is located in a human body. The housing encloses a first microprocessor, a first information processing unit, a power supply unit, and a wireless transmitter which transmits data signals to the wireless receiver in the in vitro blood sensor unit.

An in vivo first pressure sensor is located inside of a human body. An input end of the in vitro first pressure sensor contacts a human blood vessel to record and then transmits blood pressure information to a first information processing unit. The blood pressure information is then transmitted from the first information processing unit to the first microprocessor. The first microprocessor passes the blood pressure information through a wireless transmitter to a wireless receiver located outside the human body.

A power supply unit is connected to the in vivo first pressure sensor, the first information processing unit, and the wireless transmitter. The power supply unit has a plurality of micro batteries. The implantable first pressure sensor is a miniature pressure sensor, and the first information processing unit includes a signal amplifier.

The above technical solution has several beneficial effects. The above technical solution has a simple structure which utilizes an innovative integrated circuit approach constructing all major function sections into one SOC (System On Chip) type device. The SOC device includes different sensors, preamplifiers, AD/DA converters, RF transmission and receiving circuits, etc. The SOC device can be implanted into the human body through surgery and can remain in the human body for a long time without affecting the normal life of the human being. The in vivo SOC device provides true and accurate information, and it is conveniently monitored from receiving devices located external of the human body.

The above technical solution has several other beneficial effects. A signal finishing circuit is used to eliminate noise and to amplify the electrocardiographic signal. The ECG signal is then input to a central information processing unit and is classified to provide a risk level for the patient. The ECG signal and the risk level may then be displayed on a display screen, so that the ECG signal and the risk level can be easily viewed and detected problems may be treated in a timely manner. After sampling of the original electrocardiographic waveform data, detected ECG waveform data is compared with prior ECG waveform data to identify whether portions of the waveform data are distorted. The portions of the waveform data which are distorted are identified so as to avoid using inaccurate ECG data and possible missed diagnoses due to display medium resolution suppression, which improves the accuracy of diagnoses through ECG.

The above technical solution also has the following listed advantages. The invention collects various human physiological index data in real time through a measurement and human body data collecting unit which utilizes multiple innovative integrated electrical, optical, acoustic, pyroelectric and millimeter wave sensors. Sensed data is input to a central information processing unit and to a data storage unit. The data storage unit will also store the human physiological index data. Standard ranges of values for the human physiological index data are stored. The central information processing unit compares and analyzes the ranges of physiological data detected to the standard range value of the stored human physiological indexes, and makes a preliminary health diagnosis. The data is wirelessly transmitted to a remote communication module which may store the data on a cloud server. The disclosed invention preferably utilizes an advanced large-scale integrated circuit to integrate multiple human physiological index detection functions on one instrument which incoporates a central information processing unit to analyze and obtain a preliminary health diagnosis.

Although the preferred embodiments have been described in detail, it should be understood that various changes, substitutions, and alterations can be made therein without departing from the spirit and scope of the present disclosure as defined by the appended claims.

What is claimed is:

1. An intelligent portable medical instrument, comprising:
at least one information processing unit, a measurement and body data collection unit, and a data storage unit, wherein said at least one information processing unit and said measurement and body data collection unit, and said data storage unit are operatively connected for collecting, processing and storing living body data and are integrated into one or more semiconductor integrated circuit chips which are disposed within a living body;
said measurement and body data collection unit which are configured to collect physiological indicator data and send the collected physiological indicator data to said at least one information processing unit which stores the collected physiological indicator data in said data storage unit;
said data storage unit configured to store both the collected physiological indicator data and standard ranges of preset physiological indicators; and
wherein said at least one information processing unit compares the collected physiological indicator data with the standard ranges of preset physiological indicators, and determines a preliminary health diagnosis opinion according to an analysis of the comparison and sends the preliminary health diagnosis opinion to an vitro communication module.

2. The intelligent portable medical device according to claim 1, wherein said measurement and body data collection unit comprises a body temperature sensor unit, a blood pressure sensor unit, a biological brain electrical sensor unit, a biochemical sensor unit, and a bio-cardiac sensor.

3. The intelligent portable medical device according to claim 2, wherein said body temperature sensor unit consists of an infrared sensor section which contains integrated optical and pyroelectrical sensor arrays;
said integrated optical and pyroelectrical sensor arrays are made using a device structure which includes a preamplifier, an Analog to Digital (AD) conversion section, an information processing section, and a signal transmission section.

4. The intelligent portable medical instrument according to claim 2, further comprising:
said blood pressure sensor unit being composed of an in vivo blood pressure sensor unit which is operably connected to said at least one central processing unit; and
said in vivo blood pressure sensor unit includes an in vivo first pressure sensor, one of said at least one information processing units being disposed in vivo in the body to define an in vivo information processing unit, and said in vivo first pressure sensor and said in vivo information processing unit are configured to collect blood pressure information.

5. The intelligent portable medical instrument according to claim 4, further comprising:
a wearable blood pressure measurement set having an in vitro blood pressure sensor unit which includes an in vitro light emitting diode array, an in vitro optical sensor array, an in vitro piezoelectric device and sensor array, and an in vitro acoustic sensor array, wherein said in vitro light emitting diode array, said in vitro optical sensor array, said in vitro piezoelectric device and sensor array, and said in vitro acoustic sensor array are integrated into a second semiconductor integrated circuit chip.

6. The intelligent portable medical instrument according to claim 2, further comprising:
said biological brain electrical sensor having an in vivo biological brain electrical sensor unit, wherein said in vivo biological brain electrical sensor includes an in vivo capacitive sensor array, an in vivo optical sensor array, an in vivo piezoelectric sensor array, and an in vivo an acoustic sensor array which have been integrated into one of said one or more semiconductor integrated circuit chips; and
an in vitro bio-encephalographic sensor unit which includes an in vitro multi-terminal EEG electrode and an in vitro pressure sensor, with said in vitro multi-terminal EEG electrode collecting brain electrical information, and said in vitro pressure sensor collecting the contact pressure of said in vitro multi-terminal EEG electrodes contacting the living body.

7. The intelligent portable medical instrument according to claim 2, further comprising:
said biological brain electrical sensor includes an in vivo bio-encephalographic sensor having an in vivo multi-terminal EEG electrode and an in vivo second pressure sensor, wherein said in vivo multi-terminal EEG electrode is connected to said in vivo second pressure sensor;
said in vivo multi-terminal EEG electrode is configured to collect brain electrical information, and said in vivo second pressure sensor is configured to collect second contact pressure information of said in vivo multi-end EEG electrode and a second respective body contact portion; and
said in vivo multi-end EEG electrode and said in vivo second pressure sensor are respectively connected to said at least one information processing unit which receives and processes the brain electrical information and said second contact pressure information.

8. The intelligent portable medical instrument according to claim 2, further comprising:
said bio-cardiac sensor unit is composed of an in vivo bio-electrocardiographic sensor unit, wherein said in vivo bio-electrocardiographic sensor unit includes an in vivo multi-terminal ECG electrode, an in vivo third pressure sensor, and said in vivo multi-terminal ECG electrode is connected to said in vivo third pressure sensor;
said in vivo multi-terminal ECG electrode is configured to collect body electrocardiographic information, and said in vivo third pressure sensor is configured to collect third in vivo contact pressure information of said in vivo multi-terminal ECG electrode and a third respective body contact portion; and
said in vivo multi-terminal ECG electrode and said in vivo third pressure sensor are respectively connected to said at least one information processing unit which receives and processes said body ECG information from said in vivo multi-terminal ECG electrode and said third in vivo contact pressure information transmitted from said third in vivo pressure sensor.

9. The intelligent portable medical instrument according to claim 8, further comprising:
an in vitro bio-electrocardiographic sensor unit, wherein said in vitro bio-electrocardiographic sensor unit includes a multi-terminal in vitro ECG electrode, a second in vitro pressure sensor, said multi-terminal ECG electrode is connected to said second in vitro pressure sensor, and said multi-terminal in vitro ECG electrode is configured to collect body ECG information, said second in vitro pressure sensor is configured to collect second in vitro contact pressure information of contact between a contact end of said in vitro multi-end ECG electrode and the body.

10. The intelligent portable medical instrument according to claim 2, further comprising:
said bio-cardiac sensor including an in vivo capacitive sensor array, an in vivo optical sensor array, an in vivo piezoelectric sensor array, and an in vivo acoustic sensor array which have been integrated into one of said one or more semiconductor integrated circuit chips;
said in vivo capacitive sensor array and said in vivo piezoelectric sensors array are used to detect conduction signals from a heart, such as transmit signals of the sinoatrial node (SA) and atrioventricular node (AV node); and
said in vivo acoustic sensor array is utilized to detect critical acoustic signals from heart, such as the heart beat rate (BPM- Beats Per Minute), characteristics of coronary artery, cardiac muscle contraction, to increase a detection sensitivity, and an acoustic radar system is included in said acoustic sensor array.

11. The intelligent portable medical instrument according to claim 2, further comprising said biochemical sensor unit having an in vivo biochemical sensor unit includes an in vivo implantable multi-end biochemical sensor, wherein said implantable multi-end biochemical sensor is coupled to said at least one information processing unit to transmit biochemical information of the body.

12. An intelligent portable medical instrument according to claim 2, further comprising said in vivo blood glucose sensor unit including an implantable blood glucose sensor, wherein said in vivo blood glucose sensor is configured to collect blood glucose information is connected to said at least one information processing unit receives and processes the blood glucose information of the body.

13. An intelligent portable medical instrument according to claim 2, further comprising:
said in vitro blood glucose sensor including an intelligent signal source chip containing wide optical spectrum Light Emitting Diode (LED) Array and millimeter wave generating integrated circuit;
a wide spectrum optical sensor array and a millimeter wave receiving integrated circuit which is consists of signal preamplifier, AD (Analog To Digital) & DA (Digital To Analog) converter, and a central signal processing module; and
wherein a portion of living body, such as earlobe, finger, or hand, would be placed in between the intelligent signal source and the wide spectrum sensor array, and the optical and millimeter wave signal that is passing through the living body will be received by the wide spectrum optical sensor array and a millimeter wave receiving integrated circuit and analyzed by said at least one processing unit.

14. The intelligent portable medical device according to claim 2, further comprising:
an in vitro blood pressure sensor unit having an in vitro wireless receiver;
an in vivo blood pressure sensor which includes a housing located within the living body, wherein said housing encloses said at least one information processing unit, a in vivo power supply unit, a in vivo wireless transmitter; and
said in vivo first pressure sensor is located within the body casing, and the input end of said in vivo first pressure sensor contacts a blood vessel to detect blood pressure information of the living body, and an output end of said in vivo first pressure sensor transmits the blood pressure information of the body to said at least one information processing unit which is transmits the processed blood pressure information to said in vivo wireless transmitter to said in vitro wireless receiver outside the living body.

15. The intelligent portable medical device according to claim 2, further comprising:
said in vivo bio-electrocardiographic sensor unit further including an ECG microcontroller which controls said in vivo multi-terminal ECG electrode to acquire a signal;
said ECG microcontroller having a communication unit which communicates with said at least one information processing unit, an electrocardiographic signal acquisition unit, a second microprocessor which connected to said communication unit and to said electrocardiographic signal acquisition unit, wherein said second microprocessor is configured by signal acquisition;
said signal acquisition unit including a signal input interface, a signal output interface, and a signal sorting circuit, wherein said signal sorting circuit is respectively connected to said signal input interface and said signal output interface, and said signal sorting circuit is configured to perform noise elimination and amplification processing on said collected ECG signal, and then output by said signal output interface;
said at least one information processing unit includes an ECG signal discriminating unit, and said ECG signal discriminating unit is configured to analyze the ECG signals and classify according to different risk levels; and
said intelligent portable medical device further includes a display screen for displaying ECG signals and the different risk level.

16. An intelligent portable medical instrument, comprising:
at least one information processing unit, a measurement and body data collection unit, and a data storage unit, wherein said at least one information processing unit and said measurement and body data collection unit, and said data storage unit are operatively connected for collecting, processing and storing living body data and are integrated into a semiconductor integrated circuit chip which is disposed within a living body;
said measurement and body data collection unit configured to collect physiological indicator data and send the collected physiological indicator data to said at least one information processing unit which stores the collected physiological indicator data in said data storage unit;
said data storage unit configured to store both the collected physiological indicator data and standard ranges of preset physiological indicators;
wherein said at least one information processing unit compares the collected physiological indicator data with the standard ranges of preset physiological indicators, and determines a preliminary health diagnosis opinion according to an analysis of the comparison and sends the preliminary health diagnosis opinion to an vitro communication module;
wherein said measurement and body data collection unit comprises a body temperature sensor unit, a blood pressure sensor unit, a biological brain electrical sensor unit, a biochemical sensor unit, and a bio-cardiac sensor.

17. An intelligent portable medical instrument according to claim 16, further comprising:
packaging providing an enclosure in which said at least one information processing unit, said measurement and body data collection unit, and said data storage unit are disposed;
said enclosure having one or more open windows formed therein to expose first portions of said measurement and body collection unit, and wherein at least part of said enclosure is formed dielectric material to expose second portions of said measurement and body collection units; and
wherein said first portions of said measurement and body collection unit includes a piezoelectric array, an acoustic array, and a capacitive sensor array which are disposed adjacent to said one or more open windows, and said second portions of said measurement and body collection unit further includes an optical sensor array, a millimeter wave IC, and a wireless transmitter disposed adjacent to said dielectric material.

18. The intelligent portable medical device according to claim 16, further comprising:
said body temperature sensor unit including of an infrared sensor section which contains integrated optical and pyroelectrical sensor arrays, wherein said integrated optical and pyroelectrical sensor arrays are made using a device structure which includes a preamplifier, an Analog to Digital (AD) conversion section, an information processing section, and a signal transmission section;
said blood pressure sensor unit being composed of an in vivo blood pressure sensor unit which is operably connected to said at least one central processing unit, wherein said in vivo blood pressure sensor unit includes an in vivo first pressure sensor, one of said at least one information processing units being disposed in vivo in the body to define an in vivo information processing unit, and said in vivo first pressure sensor and said in vivo information processing unit are configured to collect blood pressure information;
said biological brain electrical sensor having an in vivo biological brain electrical sensor unit, wherein said in vivo biological brain electrical sensor includes an in vivo capacitive sensor array, an in vivo optical sensor array, an in vivo piezoelectric sensor array, and an in vivo an acoustic sensor array which have been integrated into said semiconductor integrated circuit chip;
an in vitro bio-encephalographic sensor unit which includes an in vitro multi-terminal EEG electrode and an in vitro pressure sensor, with said in vitro multi-terminal EEG electrode collecting brain electrical information, and said in vitro pressure sensor collecting the contact pressure of said in vitro multi-terminal EEG electrodes contacting the living body;
said biological brain electrical sensor includes an in vivo bio-encephalographic sensor having an in vivo multi-terminal EEG electrode and an in vivo second pressure sensor, wherein said in vivo multi-terminal EEG electrode is connected to said in vivo second pressure sensor;
said in vivo multi-terminal EEG electrode is configured to collect brain electrical information, and said in vivo second pressure sensor is configured to collect second contact pressure information of said in vivo multi-end EEG electrode and a second respective body contact portion;
said in vivo multi-end EEG electrode and said in vivo second pressure sensor are respectively connected to said at least one information processing unit which receives and processes the brain electrical information and said second contact pressure information;
said bio-cardiac sensor unit is composed of an in vivo bio-electrocardiographic sensor unit, wherein said in vivo bio-electrocardiographic sensor unit includes an in vivo multi-terminal ECG electrode, an in vivo third pressure sensor, and said in vivo multi-terminal ECG electrode is connected to said in vivo third pressure sensor;
said in vivo multi-terminal ECG electrode is configured to collect body electrocardiographic information, and said in vivo third pressure sensor is configured to collect third in vivo contact pressure information of said in vivo multi-terminal ECG electrode and a third respective body contact portion;
said in vivo multi-terminal ECG electrode and said in vivo third pressure sensor are connected to said at least one information processing unit which receives and processes said body ECG information from said in vivo multi-terminal ECG electrode and said third in vivo contact pressure information transmitted from said third in vivo pressure sensor;
an in vitro bio-electrocardiographic sensor unit, wherein said in vitro bio-electrocardiographic sensor unit includes a multi-terminal in vitro ECG electrode, a second in vitro pressure sensor, said multi-terminal ECG electrode is connected to said second in vitro pressure sensor, and said multi-terminal in vitro ECG electrode is configured to collect body ECG information, said second in vitro pressure sensor is configured to collect second in vitro contact pressure information of contact between a contact end of said in vitro multi-end ECG electrode and the body;
said bio-cardiac sensor including an in vivo capacitive sensor array, an in vivo optical sensor array, an in vivo piezoelectric sensor array, and an in vivo acoustic sensor array which have been integrated into said semiconductor integrated circuit chip;
said in vivo capacitive sensor array and said in vivo piezoelectric sensors array are used to detect conduction signals from a heart, such as transmit signals of the sinoatrial node (SA) and atrioventricular node (AV node); and
said in vivo acoustic sensor array is utilized to detect critical acoustic signals from heart, such as the heart beat rate (BPM—Beats Per Minute), characteristics of coronary artery, cardiac muscle contraction, to increase a detection sensitivity, and an acoustic radar system is included in said acoustic sensor array;
said biochemical sensor unit having an in vivo biochemical sensor unit includes an in vivo implantable multi-end biochemical sensor, wherein said implantable multi-end biochemical sensor is coupled to said at least one information processing unit to transmit biochemical information of the body;
in vivo blood glucose sensor unit including an implantable blood glucose sensor, wherein said in vivo blood glucose sensor is configured to collect blood glucose information, which is connected to said at least one information processing unit receives and processes the blood glucose information of the body.

19. The intelligent portable medical device according to claim 16, further comprising:
an in vitro blood pressure sensor unit having an in vitro wireless receiver;
an in vivo blood pressure sensor which includes a housing located within the living body, wherein said housing encloses said at least one information processing unit, a in vivo power supply unit, a in vivo wireless transmitter;
said in vivo first pressure sensor is located within the body casing, and the input end of said in vivo first pressure sensor contacts a blood vessel to detect blood pressure information of the living body, and an output end of said in vivo first pressure sensor transmits the blood pressure information of the body to said at least one information processing unit which is transmits the processed blood pressure information to said in vivo wireless transmitter to said in vitro wireless receiver outside the living body;
said in vivo bio-electrocardiographic sensor unit further including an ECG microcontroller which controls said in vivo multi-terminal ECG electrode to acquire a signal;
said ECG microcontroller having a communication unit which communicates with said at least one information processing unit, an electrocardiographic signal acquisition unit, a second microprocessor which connected to said communication unit and to said electrocardiographic signal acquisition unit, wherein said second microprocessor is configured by signal acquisition;
said signal acquisition unit including a signal input interface, a signal output interface, and a signal sorting circuit, wherein said signal sorting circuit is respectively connected to said signal input interface and said signal output interface, and said signal sorting circuit is configured to perform noise elimination and amplification processing on said collected ECG signal, and then output by said signal output interface;
said at least one information processing unit includes an ECG signal discriminating unit, and said ECG signal discriminating unit is configured to analyze the ECG signals and classify according to different risk levels; and
said intelligent portable medical device further includes a display screen for displaying ECG signals and the different risk levels.

20. A method for operating an intelligent portable medical device to provide an ECG signal display, the method comprises the step of:
Step S1: an information processing unit receiving the original ECG waveform data transmitted by an implanted in vivo bio cardiac sensor unit and determining waveform data to be displayed that matches the resolution of the display medium;
Step S2: performing up sampling calculations on the waveform data to be displayed, and obtaining up sampled waveform data with the same sampling rate as the original electrocardiographic waveform data;
Step S3: comparing the original ECG waveform data and the up sampled waveform data, determining whether there is waveform distortion, if yes, proceeding to step S4; if not, proceeding to step S6;
Step S4: determining a range of the waveform distortion, and proceeding to step S5;
Step S5: outputting a waveform to be displayed, and identifying a waveform distortion area and that there is a loss area of the waveform presented at the current resolution; and
Step S6: outputting the waveform to be displayed.
